(19) United States
(12) Patent Application Publication
(10) Pub. No.: US 2024/0285205 A1
(43) Pub. Date: Aug. 29, 2024

(54) Devices, Systems, and Methods for Mental Health Assessment

(71) Applicant: Innsightful, Inc., Ann Arbor, MI (US)

(72) Inventor: Farzad Ehsani, Sunnyvale, CA (US)

(21) Appl. No.: 18/572,824

(22) PCT Filed: Jun. 24, 2022
(86) PCT No.: PCT/US2022/034897

§ 371 Date: Dec. 21, 2023

Related U.S. Application Data
(60) Provisional application No. 63/214,535, filed on Jun. 24, 2021.

(51) Int. Cl. A61B 5/16; A61B 5/00

(52) U.S. Cl. CPC A61B 5/165; A61B 5/4803; A61B 5/7267; A61B 5/7282

(57) ABSTRACT

A device, system, and method for assessing and managing mental health of an individual. A device is configured to evaluate data which corresponds to one or more biosignals of the individual, and to detect the intensity of both positive and negative emotional valence in the individual. The individual may have neither positive nor negative emotional valence (e.g., when at rest), only one of positive and negative emotional valence, or some amount of both. If the detected negative emotional valance surpasses a first threshold, the device or system may provide an intervention to the individual to help the individual manage stress and improve mental health. If the detected positive emotional valence surpasses a second threshold, the device or system may record information related to the individual's stress level and provide encouragement to help the individual continue the positive activity.

### CROSS REFERENCE TO RELATED APPLICATIONS

This application claims priority and benefit to Provisional Patent Application Ser. No. 63/214,535 filed on Jun. 24, 2021.

### TECHNICAL FIELD

The disclosure relates to computer program products, devices, systems, and methods for assessing mental health of an individual. More particularly, a device or system is configured to differentiate between absence and presence of stress in an individual, and if stress is present, to quantify and characterize the stress and includes an approach that may evaluate physiological data of the individual under a controlled condition and correlate the physiological data with the controlled condition, and may evaluate physiological data of the individual under an uncontrolled condition and correlate the physiological data of the uncontrolled condition with the physiological data of the controlled condition to detect, quantify, and characterize stress and mental health of the individual in the uncontrolled condition and, if necessary, to intervene with the individual to manage mental health.

### BACKGROUND

Many individuals, both in the U.S. and throughout the world, suffer from adverse mental health conditions. Currently, 70.5% of college students report their academic performance is affected by stress, including psychological stress; 53.1% are affected by anxiety, and 34.5% by depression. Student use of college counseling centers increased by an average of 30-40% from 2009 to 2015. Many college campuses have no on-campus psychiatric support, and if they do, wait times are extremely long, resulting in crisis situations for many students. According to the CDC, more than 50% of the general population will be diagnosed with a mental illness or disorder at some point in their lifetime. The most common mental health issues among college students are anxiety, depression, relationship issues, and thoughts of suicide. Unfortunately, only half receive treatment, which may be due to significant stigma around mental health and may also point to significant issues with the current mental healthcare treatment model.

Existing solutions for monitoring and treating mental health conditions are not adequately meeting the growing demand for these services. The limited availability of therapists and psychiatrists leads many patients to forego treatment altogether or even experience significant mental health crises while waiting to see a health professional. Additional factors, such as transportation cost or availability, and lack of acceptable health insurance, complicate traditional treatment approaches. The effect is a high healthcare and economic burden, on both caretakers and patients. As a result, many patients suffer for extended periods of time without receiving effective treatment.

Accordingly, there is a need for real-time automated monitoring, detecting, classifying, and intervening with individuals for the assessment and management of mental health.

### SUMMARY

The system provides an end-to-end platform to monitor and manage mental health conditions using at least one biosensor device and at least one software application. The platform can be utilized by healthcare providers for diagnosis, non-contact therapeutics, monitoring, and population management. The approaches may be scaled according to need to make better use of available healthcare resources and may be used to monitor at-risk patients, whether for mental or physical health, as well as those who have received a diagnosis and require remote or at-home monitoring and, in certain instances, intervention for health care management.

The system effectively differentiates between positive psychological stress (eustress) and negative psychological stress (distress) and quantitates the amount of stress experienced by the individual. Approaches may include a biosensor device, a biological signal (biosignal) event detection artificial intelligence (AI) engine, an automated conversation agent, real-time connectivity with health care providers, a regulatory-compliant (e.g., HIPAA-compliant) backend, or any combination thereof for real-time mental health monitoring, assessment, and, in certain instances, management and intervention.

A method for a system comprising a non-transitory machine-readable medium storing instructions that, when executed by at least one processor, cause the at least one processor to perform operations for mental health assessment, the operations having the steps of determining at least a first set of data which corresponds to at least one physiological parameter of an individual, an environment of the individual, an interaction between the individual and the environment, or any combination thereof, evaluating said least a first set of data, and detecting the presence and magnitude of positive and negative emotional valences of the individual based on the evaluation of at least the first set of data. In some embodiments, operations further include the steps of determining a second set of data which corresponds to which corresponds to at least one previous exposure of the individual to at least one known positive stressor, at least one known negative stressor, or any combination thereof, evaluating said second set of data, and wherein the evaluation of the first and second sets of data yields the positive and negative emotional valences of the individual. The operations may further include querying the individual with a conversational agent of the instructions to produce at least one query result, evaluating the at least one query result to assist the operations with the detecting the positive and negative emotional valences, and wherein the operations further include determining a magnitude of each of the positive and negative emotional valences and placing at least one data point on a V-vector model based on the magnitudes. Another step may further include determining a magnitude of each of the positive and negative emotional valences and placing at least one data point on a V-vector model based on the magnitudes.

The operations may further include providing an intervention to the individual if the negative emotional valence surpasses a first threshold of the V-vector model and recording information related to a stress level of the individual if the positive emotional valence surpasses a second threshold of the V-vector model.

A system for mental health assessment, the system for mental health assessment including at least one processor and at least one non-transitory machine-readable medium storing instructions that, when executed by the at least one processor, cause the at least one processor to perform operations for mental health assessment, the operations may include the steps of determining at least a first set of data which corresponds to at least one physiological parameter of an individual, an environment of the individual, an interaction between the individual and the environment, or any combination thereof, evaluating said least a first set of data, and detecting positive and negative emotional valences of the individual based on the evaluation of that data. The operations may further include evaluating a second set of data which corresponds to at least one previous exposure of the individual to at least one known positive stressor, at least one known negative stressor, or any combination thereof, wherein the evaluation of the first and second sets of data yields the positive and negative emotional valences of the individual.

The operations may further include querying the individual with a conversational agent of the instructions to produce at least one query result and evaluating the at least one query result to assist the operations with the detecting the positive and negative emotional valences. A further step may include determining a magnitude of each of the positive and negative emotional valences and placing at least one data point on a V-vector model based on the magnitudes. An additional step may include providing an intervention to the individual if the negative emotional valence surpasses a first threshold of the V-vector model. An additional step may include recording information related to a stress level of the individual if the positive emotional valence surpasses a second threshold of the V-vector model.

A method for assessing mental health of an individual, the steps may include determining at least a first set of data which corresponds to at least one physiological parameter of the individual, an environment of the individual, an interaction between the individual and the environment, or any combination thereof, evaluating said at least a first set of some data, detecting positive and negative emotional valences of the individual based on the evaluation of at least the first set of data, and evaluating a second set of data which corresponds to at least one previous exposure of the individual to at least one known positive stressor, at least one known negative stressor, or any combination thereof, wherein the evaluation of the first and second sets of data yields the positive and negative emotional valences of the individual. An additional step may include querying the individual to assist with the detecting the positive and negative emotional valences. Another step may include determining a magnitude of each of the positive and negative emotional valences and placing at least one data point on a V-vector model based on the magnitudes. Another step may include providing an intervention to the individual if the negative emotional valence surpasses a first threshold of the V-vector model and/or recording information related to a stress level of the individual if the positive emotional valence surpasses a second threshold of the V-vector model.

In one aspect, the system provides computer program products which comprise of at least one non-transitory machine-readable medium. This medium stores instructions that, when executed by at least one processor, cause the at least one processor to perform operations for mental health assessment, the operations comprising evaluating at least a first set of data which corresponds to at least one physiological parameter of an individual, an environment of the individual, an interaction between the individual and the environment, or any combination thereof, and detecting both positive and negative emotional valences of the individual based on the evaluation of at least the first set of data. At any moment, the person may be experiencing neither type of emotional valence (at rest) or may be experiencing only one or both types of emotional valence. The positive stress may correlate with a situation or event that may not negatively impact the individual's mental health, and the negative stress may correlate with a situation or event that may negatively impact the individual's mental health. The operations and methods of the system may be performed real-time as the biosignal data is collected, e.g., for crisis intervention and prevention, or may be performed after the collection of biosignal data. The positive and negative emotional valences may be simultaneously detected to enable real-time mental health assessment and management.

In another aspect, the system provides devices and systems comprising of at least one processor and at least one non-transitory machine-readable medium which stores instructions that, when executed by the at least one processor, cause the at least one processor to perform operations for mental health assessment and management. Generally, any suitable device or system is envisioned for implementation of the system.

In another aspect, the system provides methods for assessing mental health of an individual, involving evaluating at least a first set of data which corresponds to at least one physiological parameter of the individual, an environment of the individual, an interaction between the individual and the environment, or any combination thereof, and detecting positive and negative emotional valences of the individual based on the evaluation of at least the first set of data. The person may be experiencing neither type of emotional valence (at rest) or may be experiencing only one or both types of emotional valence. The methods may be performed by the individual, or another individual such as a healthcare provider or crisis intervention specialist.

The operations and methods may comprise evaluating a second set of data which corresponds to at least one previous exposure of the individual to at least one known positive stressor, at least one known negative stressor, or any combination thereof; wherein the evaluation of the first and second sets of data yields the positive and negative emotional valences of the individual. For example, the first set of data may relate to at least one biosignal collected in an uncontrolled condition, and the second set of data may relate to at least one biosignal collected in a controlled condition. The operations and methods may correlate physiological data of the individual under the uncontrolled condition with physiological data of the individual under the controlled condition to detect, quantify, and characterize stress and mental health in the uncontrolled condition, and, in certain instances, to provide an intervention to the individual for management or improvement of mental health.

The detecting positive and negative emotional valences of the individual may be performed by at least a classification engine of the instructions, which may be based on a suitable algorithm or approach for classification. Exemplary approaches include, but are not limited to: an artificial neural net (ANN) having one or multiple layers, a convolutional neural net (CNN), a continuous regression model such as linear or polynomial regression, a support vector machine (SVM), a random forest, and the like.

The operations may involve querying the individual with a conversational agent of the instructions to produce at least one query result, and evaluating the at least one query result to assist the operations with the detecting the positive and negative emotional valences. The conversational agent may be configured to automatically query the individual to obtain information about a biosignal event. Such queries may include asking the individual whether any stress experienced is positive, negative, or both. Such a process may, in certain instances, assist the classification engine with classifying the event and may be used to improve performance of the classification engine.

The operations may involve determining a magnitude or intensity of each of the positive and negative emotional valences, and, based on the determined magnitudes, generating and placing at least one data point on a V-vector model. The V-vector model may be used to map the emotional valences, with or without visual representation, on a two-dimensional axis representative of stress magnitude (e.g., high stress, medium stress, low stress, no stress) and sign (e.g., positive, negative, both, neutral).

The operations may involve providing an intervention to the individual if the negative emotional valence surpasses a first threshold value of the V-vector model, such as a threshold for negative stress. Some individuals may find a degree of negative stress to be tolerable and may not always need an intervention if some negative stress is experienced. Accordingly, a threshold suited to the individual may be utilized. The operations may, in addition or in the alternative, involve recording information related to a stress level of the individual if the positive emotional valence surpasses a second threshold of the V-vector model, such as a threshold for positive stress. The recorded information may be stored and retrieved by the operations and methods and presented or suggested to the individual as a potential intervention for managing negative stress associated with a subsequent biosignal event (e.g., a subsequent biosignal event that surpasses the first threshold value for negative stress). In this manner, the activity or activities suggested for future interventions, which may be based on an activity or activities previously identified as shifting stress from negative to positive, may become more relevant and effective with increased use of the system.

In another aspect, the system leverages artificial intelligence (AI) engines to solve the growing market need for continuous, real-time, and remote mental health monitoring and management. Implementations of the system may utilize multimodal objective biometrics from wearable technology, AI for detection and classification of events, and in at least some instances, the seamless integration of AI workflows with human clinician experts.

In another aspect, the system provides novel biometric-monitoring technologies which provide an effective solution for early detection of physical and mental illnesses in individuals. These technologies allow real-time analysis of biometric markers tied to stress imbalance, e.g., eustress (or positive stress) and distress (or negative stress). Paired with software applications such as mobile applications, these approaches provide solutions for not only detecting distress and rising stress loads, but also generating actionable information regarding an individual's behavioral health. As such, implementations of the system may enable just-in-time (JIT) therapeutics and may provide comprehensive approaches for monitoring and encouraging positive or balanced behavioral health, ultimately improving the health and resilience of patients and individuals.

Another object of the system is to provide computer program products, devices, systems, and methods that may be readily employed to benefit at-risk populations and scale a strained healthcare system to benefit those most in need.

Other objects, features and advantages of the system will become apparent from the following detailed description taken in conjunction with the accompanying drawings.

### BRIEF DESCRIPTION OF THE DRAWINGS

Although the characteristic features of the system will be particularly pointed out in the claims, exemplary embodiments and manners in which they may be made and used may be better understood after a review of the following description, taken in connection with the accompanying drawings, wherein like numeral annotations are provided throughout.

FIG. 1A depicts a first V-vector model, having a first positive valence slope and a first negative valence slope, illustrating two-dimensional stress detection and classification using a single threshold at a first intensity level based on the valence slopes.

FIG. 1B depicts a second V-vector model, having a second positive valence slope and a second negative valence slope, illustrating two-dimensional stress detection and classification using a single threshold at a second intensity level based on the valence slopes.

FIG. 2A depicts a V-vector model having a first threshold (for negative stress) and a second threshold (for positive stress), such that the thresholds are each parallel to the vertical axis.

FIG. 2B depicts a V-vector model having a first threshold (for negative stress) and a second threshold (for positive stress), such that the thresholds are each perpendicular to a corresponding valence axis.

FIG. 3A depicts an exemplary biosensor device next to an exemplary HRV graph.

FIG. 3B depicts one or more exemplary biosensor devices, next to an exemplary skin conductance response (SCR) graph, showing electrodermal activity (EDA) as a function of time.

FIG. 4 depicts a diagram of an exemplary end-to-end system according to the system.

FIG. 5A depicts a diagram of an exemplary method of training a device or system in a controlled environment to configure the device or system for monitoring an individual for stress in an uncontrolled environment.

FIG. 5B depicts a diagram of a method or operation of monitoring the individual for stress in the uncontrolled environment, and for evaluating detected stress against at least a first threshold for negative stress.

FIG. 5C depicts a diagram of a method or operation of monitoring the individual for stress in the uncontrolled environment, and for evaluating detected stress against at least a second threshold for positive stress.

FIG. 6 depicts a block diagram of a machine in the example form of a computer device or system, with which instructions may be executed to cause the machine to perform any one or more of the operations and/or methodologies of the system, in whole or in part, individually or in combination, in any order of events or steps.

FIG. 7 depicts a diagram of an exemplary embodiment of the system that utilizes biosignal data (e.g., ECG, EDA, respiration, accelerometer, temperature, or any combination thereof) and a convolutional neural net (CNN) for classifying events as being positive affect (PA) or negative affect (NA).

### DETAILED DESCRIPTION OF THE INVENTION

Reference is made herein to the attached drawings. Like reference numerals are used throughout the drawings to depict like or similar elements of the system. The figures are intended for representative purposes only and should not be considered limiting in any respect.

Young individuals (18-19, 20-21, and 22-25 age groups) are experiencing a substantial increase in psychological distress relative to older individuals (26-49, 50+). There exists a substantial need for innovation in mental healthcare, particularly for at least these groups. Stress may be physiological or psychological in nature, and psychological stress may be positive (e.g., eustress) or negative (e.g., distress). Positive has a positive impact on the patient's wellbeing, while distress has a negative impact. The system provides novel and valuable approaches for distinguishing between positive psychological stress associated with challenge states (eustress) and negative psychological stress associated with threat states (distress).

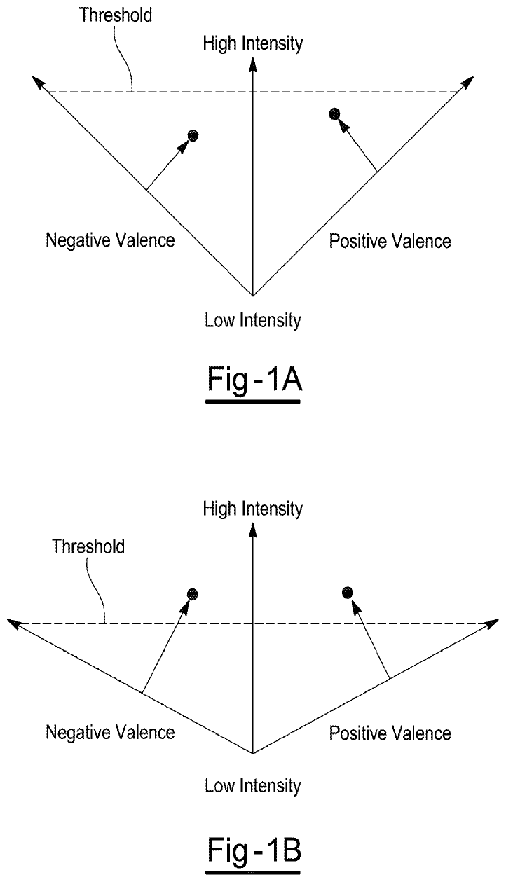

Referring now to FIG. 1A and FIG. 1B, there are depicted a first V-vector model, having a first positive valence slope and a first negative valence slope, illustrating two-dimensional stress detection and classification using a single threshold at a first intensity level based on the valence slopes (FIG. 1A), and a second V-vector model, having a second positive valence slope and a second negative valence slope, illustrating two-dimensional stress detection and classification using a single threshold at a second intensity level based on the valence slopes (FIG. 1B). The system provides approaches for determining a magnitude of each of the positive and negative emotional valences and placing at least one data point on a V-vector model based on the magnitudes for evaluation of mental health. The system provides approaches for distinguishing between three different states which are related to one another in two different dimensions. The three states are negative stress, positive stress, and no stress. The presence or absence of stress (which may be referred to as the intensity of the stress) is embodied by the vertical axis, and the nature of the stress (negative or positive) is embodied by the left and right axes. Because negative stress and positive stress are each a type of stress, the left and right axis each contains a vertical portion, and positive and negative stress do not cancel each other out to form no stress, but rather, various degrees of neutral stress may be experienced if both positive and negative forms of stress are present in the individual. For example, a person maybe both excited about doing an activity and afraid at the same time.

One or more stress thresholds may be utilized with operations and methods of the system to trigger an intervention with the monitored individual, record information related to the stress level for future retrieval, or both. As a non-limiting example, if stress levels exceed a threshold for negative stress, an intervention may be provided to help the individual manage stress. Similarly, as a non-limiting example, if stress levels exceed a threshold for positive stress, the circumstances associated with the positive stress may be noted and stored for future use in providing interventions for exceeding the negative threshold. In certain instances, the threshold(s) may raise or lower along the intensity axis according to the individual's tolerance for stress or a particular type of stress (e.g., positive, or negative). As such, while the threshold is shown in the figures as a continuous horizontal (dotted) line, in other instances it may be irregular or discontinuous. In addition, as the slopes of the valence axes may be different between individuals, the shape of the V-vector may be specific to a particular individual. Similarly, because the slopes of the valence axes may change for a particular individual as a result of the passage of time, the individual's adaptation to circumstances, the individual's perception of various intensities, or other experiences, the shape of the V-vector may be specific to a particular timeframe or circumstance of the life of the individual. In addition, while symmetric V-vectors are shown as examples in the present disclosure, the system may, in addition or in alternative, provide approaches for making and using asymmetric V-vectors for mental health assessment, depending on the individual's magnitude of respective stresses.

Because the slopes of the valence axes may vary among individuals and among different timeframes of an individual's life, a particular biosignal event experienced by a first individual may be more tolerable compared to the same biosignal event experienced by a second individual. For example, if FIG. 1A relates to the first individual and FIG. 1B relates to the second individual, and if the data points shown in FIG. 1A and FIG. 1B are understood as having originated from identical biosignal data, because these individuals have different baseline stress levels, the severity of the event is relatively lower (FIG. 1A) or relatively higher (FIG. 1B) depending on that individual's baseline stress and threshold level(s). In some cases, as shown, data points that are below the stress threshold for the first individual may be above the threshold for the second individual. As a result, the severity of the event that produced these data points may be more accurately evaluated by measuring a distance from the nearest valence axis (e.g., negative valence or positive valence) to the corresponding data point, as shown, rather than measuring along the intensity axis.

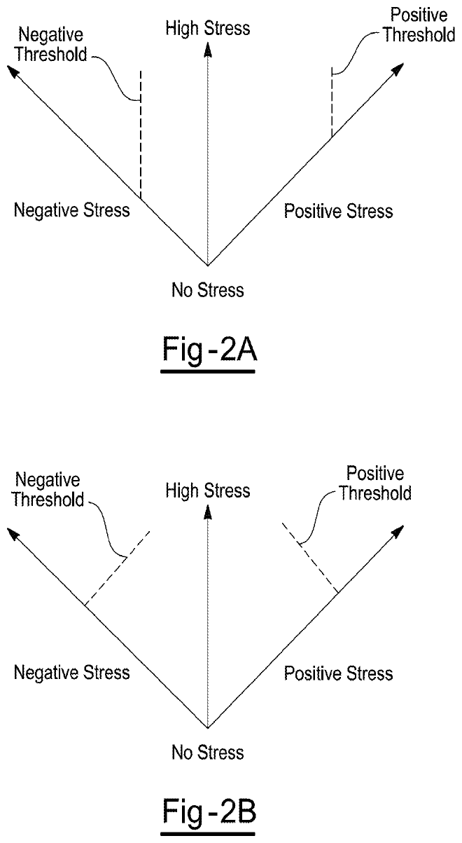

Referring now to FIG. 2A and FIG. 2B, there are depicted a V-vector model having a first threshold (for negative stress) and a second threshold (for positive stress), such that the thresholds are each parallel to the vertical axis (FIG. 2A) and a V-vector model having a first threshold (for negative stress) and a second threshold (for positive stress), such that the thresholds are each perpendicular to a corresponding valence axis (FIG. 2B). In FIG. 2A, the shown thresholds are vertical thresholds which demarcate stress levels which may relate to the most (negative) stress acceptable for the individual without initiating an intervention or the most (positive) stress allowed without necessarily recording information related to the (positive) stress level for future reference. These thresholds may be determined statistically, for example, by developing an understanding of what the individual believes is their limit for distress. In the shown embodiment, the magnitude or amount of stress may have little or no impact in determining whether additional steps are necessary; if the stress becomes sufficiently negative, intervention may be initiated, and if the stress becomes sufficiently positive, information may be recorded accordingly. In FIG. 2B, the shown thresholds are diagonal thresholds which demarcate stress levels which may represent the most negative and highest stress acceptable for the individual without initiating the intervention (negative threshold) and the most positive and highest stress observable before additional steps are taken to store information about the positive stress for possible later use. In the shown embodiment, the magnitude or amount of stress, in addition to whether the stress is sufficiently negative or positive, may more significantly affect whether such additional steps are necessary. In the shown figures the thresholds are depicted as straight lines, but any shape may be used for the threshold, including any irregular shape, if it is determined to be effective for a particular individual, group of individuals, or timeframe.

system is configured to reduce psychological distress and anxiety with a wearable biosensor device and AI-based continuous monitoring. Exemplary biosignals which may be monitored and utilized as physiological data, either alone or in combination, include but are not necessarily limited to heart rate variability (HRV), electrodermal activity (EDA), SpO2, motion, and body temperature. Any suitable sensor or combination of sensors (e.g., sensor(s) that measure cytokine or other chemical elements in sweat) may assist with improving both the specificity and sensitivity of an algorithm of the system.

Because the individual may be continuously monitored, the system may be configured to collect data that pertains to a resting state as well as an aroused state. The resting state may correlate with a relatively lower frequency or number of events detected by the system, and the aroused state may correlate with a relatively higher frequency or number of events detected by the system. In embodiments, if a classification engine of the system needs assistance classifying an event as positive-signed or negative-signed, the individual may be queried (e.g., by an automated conversational agent or a caretaker, such as a physician) to determine whether the stress event involves positive stress, negative stress, or both. This may occur at the time of the arousal. One method that may be used to characterize the stress verbally may involve using a Perceived Stress Scale (PSS) questionnaire. The PSS questionnaire is a measure of the degree to which situations are considered by an individual as stressful. Questions of the PSS questionnaire are designed to determine how uncontrollable, unpredictable, and overloaded respondents feel with respect to situations experienced in life.

As a non-limiting example, if a user becomes aroused through exercise or ordinary physical exertion, the system may ask the user whether this arousal represents positive stress or may determine this without user input, for example, by computationally evaluating physiological data of the arousal. These questions may be explicit or implicit. In embodiments, by collecting and analyzing enough information that pertains to the individual, the system may learn which stress events are detrimental and require intervention, which stress events are mildly detrimental and may not require intervention, and which stress events are not detrimental and do not require intervention. In this manner, the system may adapt, change, or evolve with a user to learn whether and how to best intervene for delivery of care.

The system provides at least one biosensor device (e.g., a wrist worn wearable device) and at least one software application which may be deployed on an individual's device, such as a mobile device, on the biosensor device, or on another device such as a networked server. The system provides an end-to-end way to effectively detect and alleviate distress and anxiety events at the time of (or shortly after) occurrence. More specifically, the biosensor device may capture heart rate variability and electrodermal activity, as well as other passively collected data sources, as biometric inputs to detect negative stress. In embodiments, artificial intelligence (AI) algorithms may be trained and validated to detect negative stress based on biosensor physiological data. Once detected, the software application may provide a combination of just-in-time activities, such as pre-selected positive activities and interventions, to mitigate events in real-time to reduce anxiety before it begins or worsens. Furthermore, the system provides a speech interface that may utilize AI and natural language processing (NLP) to “interview” the individual, collecting subjective data that relates to that individual. These interviews pinpoint the stressful event and specific triggers. Results of these AI/NLP highly personalized interviews and biometrics may inform short-term and long-term strategy selection for cognitive behavioral therapy (CBT) or related interventions to help individuals better manage negative stress and anxiety over time.

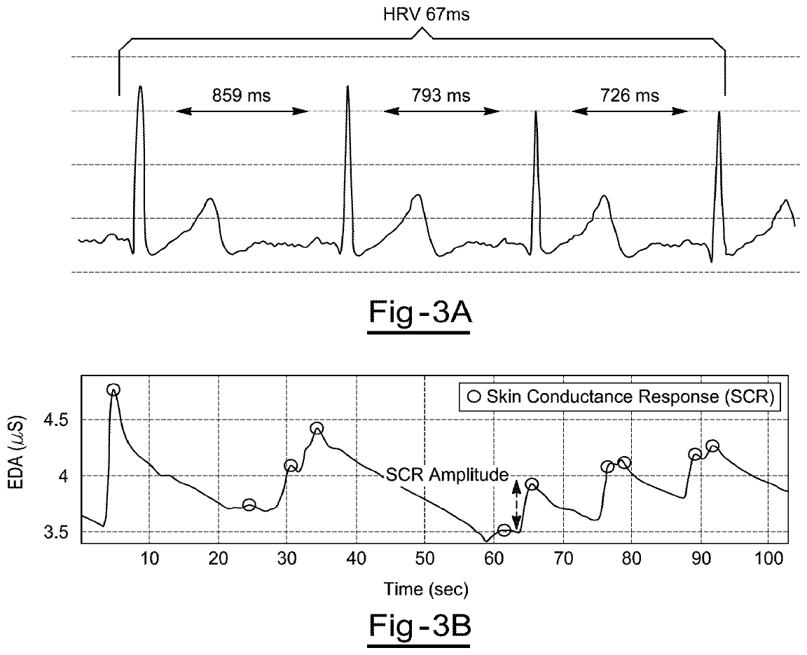

Referring now to FIG. 3A, there is depicted an exemplary biosensor device next to an exemplary HRV graph. Fluctuations in heart rate result from complex, non-linear interactions between a plurality of different physiological systems, but the autonomic nervous system (ANS) is most prominent and HRV is a function of the heart-brain interaction. The parasympathetic and sympathetic nervous systems regulate the body at rest and during fight-or-flight scenarios, respectively, and significantly impact HRV, making HRV a reflection of a person's physiological and psychological wellbeing. Specifically, HRV is a reliable parameter for analyzing the chronic shift toward increased sympathetic and decreased parasympathetic activity typical of autonomic imbalance. In another aspect, HRV is an indicator of better general health status, self-regulatory capacity, adaptability, and emotional and stress resilience. Specifically, low HRV is an indicator of abnormal and insufficient adaptation of the ANS and may be associated with harmful health events, especially when sustained for a prolonged period.

In embodiments, the system uses HRV as a neurocardiac function biomarker to detect and classify psychological stress, anxiety disorders, and depression. The biosensor device measures HRV, e.g., on a wrist of the wearer, through photoplethysmography (PPG) and feeds the acquired signal into a device or system comprising software configured to calculate parameters that are time- and frequency-based. HRV may be measured over different time periods, e.g., from less than 5 minutes to 24-hours, which may make the disclosed approaches particularly useful for facilitating clinical HRV assessments. Such approaches may be based on non-linear methods which detect and measure peculiar aspects of cardiac rhythm dynamics that are not considered by linear techniques.

There is depicted one or more exemplary biosensor devices next to an exemplary skin conductance response (SCR) graph, showing electrodermal activity (EDA) as a function of time. Sweat gland activity influences skin conductance and thermoregulation. Sweat glands are regulated by the autonomic nervous system as well and are modulated during the fight-or-flight response. In particular, the sympathetic branch stimulates sweat glands and elevates sweating. Since the number of active sweat glands increases with sympathetic activation, skin conductivity is proportional to sweat secretion, and a change in skin conductance at the surface reflects the sympathetic activity and provides a non-invasive and sensitive measure of sympathetic activity. EDA, as part of a technology that automatically recognizes psychological stress, anxiety, and depression, is suggested to be a powerful tool both in clinical settings and in daily life.

EDA was traditionally measured by sensors placed on high-density sweat gland areas (e.g., fingers, palms, etc.). The system of this embodiment allows EDA signal collection outside of the laboratory setting with a biosensor device that can continuously measure EDA with designs that integrate sensor contacts. EDA can be assessed by measuring the electrical conductance, resistance, impedance, or admittance of the skin via endosomatic and exosomatic methods to distinguish the tonic and phasic components of the electrical signal. EDA's tonic component, skin conductance level (SCL), is related to the slowly varying skin conductance level, and corresponds to baseline level of skin conductance. SCL may be computed as a mean of several measurements taken during a specific non-stimulation rest period. Thus, SCL is slowly changing and measures general psychophysiological activation (that can vary substantially among individuals). The phasic component is the rapidly fluctuating part of EDA which corresponds to a response to a specific and discrete stimulus, such as when the sudomotor nerve is activated. Generally, the increase in skin conductance starts 1 to 4 seconds after stimulus exposure, and persists for 1 to 3 seconds, allowing different amplitudes to be easily measured. Difficulties arise when responses occur continually and close together in time, making it difficult to distinguish between individual peak events. Accordingly, the present disclosure additionally relates to novel approaches for extracting overlapping peaks and filtering noise artifacts. In embodiments, these approaches measure raw conductance and apply an algorithm to extract the tonic and phasic components into separate features. For example, Phasic Peaks, Max and Variance, Tonic Peaks, Mean and Variance, and the like are variables which may be sorted. In this manner, the peak events can be characterized, and nervous system activity monitored and quantified.

In embodiments, the system involves a functionality configured to detect and classify mental health events, including but not necessarily limited to anxiety, distress, and depression, with a high degree of accuracy. The system may utilize a rule-based algorithm for distress detection based on both EDA and HRV using a ML algorithm. In embodiments, a Tree-based Pipeline Optimization Tool (TPOT) may be utilized, which is an automated machine learning tool that optimizes machine learning pipelines using genetic programming, to build the best model. This mix of methods will overcome the shortcomings associated with each individual respective method. In embodiments, the system may accurately detect when the individual is in one of several states, including a calming task, a negative stressor task, and a positive stressor task.

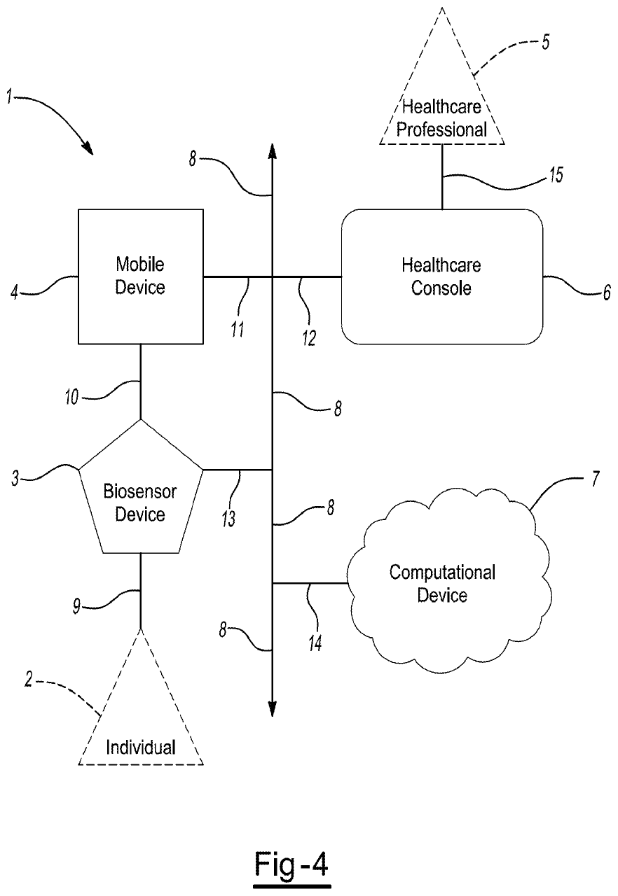

Referring now to FIG. 4, there is depicted a diagram of an exemplary end-to-end system, according to the embodiment. The system provides a method 1 for stress detection and management, comprising one or more devices (e.g., 3, 4, 6, 7) configured to monitor physiological data of an individual 2 who may be wearing or interacting with a device of the one or more devices. The system 1 may be configured to detect a biosensor event and to characterize the event as correlating with no stress, positive stress, negative stress, or both. In the shown embodiment, the individual 2 may connect with a healthcare professional 5 or another individual capable of aiding the individual 2. In this manner, the individual 2 may communicate with the healthcare professional 5 if needed for delivery of care to the individual 2.

Connections 8, 9, 10, 11, 12, 13, 14, and 15 may include, but are not limited to, any known wired or wireless connection type suitable for transmission of data, such as digital data. Generally, these connections are necessary to enable components of the system (e.g., a biosensor device 3, a mobile device 4, a healthcare console 6, and a computational device 7) to intercommunicate. Exemplary connection types that may be suitable for one or more of connections 8, 9, 10, 11, 12, 13, 14, and 15 include ethernet or other wired connection type, WiFi® connectivity, Bluetooth® connectivity, or other electromagnetic or radio wave connectivity as needed according to an embodiment.

Still referring to FIG. 4, in the shown embodiment the individual 2 is wearing a biosensor device 3 and is operably connected thereto by connection 9. Connection 9 may be representative of a physical connection, as may be utilized with attachment of the biosensor device 3 to the wearer 2, but particularly, connection 9 may be representative of an operable connection configured to enable observation of the biological property of the wearer 2 by the biosensor device 3. For example, connection 9 may include, but is not necessarily limited to, a wire connected to a skin conductance sensor for EDA measurement, an optical connection for performing pulse oximetry or heart rate detection for HRV measurement, or a combination thereof. In like manner, and consistent with embodiments of the connection 9, the biosensor device 3 may be configured to read one or more biological properties to obtain physiological data, including but not necessarily limited to a heart rate (HR), a heart rate variability (HRV), an oxygen saturation (SpO2), an electrodermal activity (EDA), a breathing rate (BR), a body temperature (BT), a movement, and any combination thereof. In embodiments, the biosensor device 3 involves an optical data acquisition system that is configured to perform pulse oximetry (for SpO2 determination) and heart rate detection (for HR or HRV determination). In embodiments, the biosensor device 3 may be configured for placement on a wrist, a finger, or an ear of the wearer. By measuring at these locations, the blood oxygen level may be optically determined, along with other parameters accessible from these body parts, according to need.

In the shown embodiment, the one or more devices involves a mobile device 4 and the biosensor device 3, wherein the mobile device 4 is operably connected to the biosensor device 3 by operable connection 10. Operable connection 10 may include any connection suitable for electronic data transmission, for example, a wired connection, or one or more wireless data connections. In embodiments, the biosensor device 3 is operably connected to the mobile device 4 by the operable connection 10, wherein the operable connection 10 is comprised of a Bluetooth® wireless connection. In this manner, the biosensor device 3 is sufficiently compatible with existing mobile devices.

In the shown embodiment, the mobile device 4 is connected to a network 8, such as the world-wide web or internet, by an operable connection 11. Operable connection 11 may include any connection type suitable for data transmission, including a wired connection or a wireless connection. Operable connection 11 may be direct or indirect, and may proceed through any number of routers, cell towers, satellites, and the like. In this manner, the system may be configured for use with any of a variety of different network designs or topologies, according to need and availability of network infrastructure in an embodiment. As ordinarily understood, network 8 may include but is not necessarily limited to a trans-continental network such as the world-wide web or internet. Network 8 may include any number of connection points, as would be understood by a person having ordinary skill in the art.

In the shown embodiment, the biosensor device 3 is operably connected to the network 8 by operable connection 13. In embodiments, the biosensor device 3 may contain one or more networking functionalities, such as one or more wireless transceivers, to enable this functionality. In embodiments, the operable connection 10, the mobile device 4, and the operable connection 11 may not be needed for full functionality of the system 1. For example, in embodiments, the biosensor device may include a non-transitory machine-readable medium containing data storage and machine-executable instructions thereon which may effectively replace the need for the mobile device 4. In this manner, the number of components of the system 1 may be reduced or maintained at a level manageable for a particular need, such as an individual 2 that may not utilize the mobile device 4 to use the system.

In embodiments, the system 1 comprises the computational device 7, and the computational device 7 may involve a networked server, operably connected to the mobile device 4 and configured to execute a machine learning (ML) algorithm to evaluate a detected event. The computational device 7 may be operably connected to the mobile device 4 by the network 8, and by at least network connection 14. Generally, network connection 14 may be any wired or wireless connection suitable for data transfer, and in this manner, the computational device 7 may be connected to the network 8 and transmit and receive data as needed.

In embodiments, one or more devices, such as the computational device 7, may include a module for event-driven activities, a module for natural language understanding (NLU), a module for speech to text (ASR), or a combination thereof. These modules may be provided as executable computer algorithms, in the form of machine-readable instructions stored in a non-transitory machine-readable medium of the computational device 7. As the system 1 operates, requests from the mobile device 4 or the biosensor device 3 may be processed and responses produced accordingly. The computational device 7 may include any suitable server, hard drive, or cloud-based networking infrastructure or equivalent combination of computational hardware which effectively enables cloud-based computing and communication.

In embodiments, one or more of the devices (3, 4, 6, 7) involves a history of the individual 2, wherein the history involves data that pertains to a previous symptom, a previous intervention, an efficacy of a previous intervention, or a combination thereof. In this manner, the system 1 may learn from previous interactions with the individual 2 to anticipate symptoms and improve interventions. In embodiments, the computational device 7 may include a data structure for storage and retrieval of therapy lessons, and a data structure for storage and retrieval of user data, sensor data, audio files, chat logs, or a combination thereof. In this manner, the history of the individual may be stored on the computational device.

The healthcare console 6 may be comprised of any suitable networked computing device, such as a mobile device (i.e., mobile phone, tablet, etc.), or a personal computer or workstation. The healthcare console 6 may be configured to enable the healthcare professional 5, such as a doctor, a psychiatrist, a therapist, and the like, to chat with the individual 2 and provide therapy recommendations based on a current situation in view of the history of the individual 2. The healthcare console 6 may be connected to the network 8 by connection 12, which may be a wired or a wireless data transfer connection. The individual may also directly input data into the mobile device.

In embodiments, the AI engine runs on the smartphone and executes time series event detection using a classification engine. The final step is to classify the event, if possible, so that predetermined logic can be executed. If there is any doubt of the classification, then the individual may be queried to identify the type(s) of stress correlated with the event. This approach allows the logic to be easily trained on tagged data, and a limited amount of verification may be necessary. In embodiments, the automated conversational agent may provide tagging of events, e.g., from the individual, which can facilitate training of the model(s) of the system and provide customization of the system to the individual or a group of individuals. In this manner, the system may be more efficiently and effectively trained, and may be smaller, more lightweight, and generally improved compared to data-heavy approaches.

The classification engine may be configured to capture multiple inputs and produce secondary data sets (e.g., combinations) that rapidly increase the complexity of the calculations. The classification engine may be assisted by the automated conversation agent, with input from the individual, to classify the event as needed. In embodiments, the classification engine may include a support vector machine (SVM), which is a non-neural network algorithm for manipulating data by adding dimensional information to classify data. In embodiments, the classification engine may include a random force algorithm. In embodiments, the system may provide a time-series analysis.

In embodiments, the system provides a simple, user-friendly interface for communicating with a health care provider, with either live chat or with the AI-driven mode. This enables direct communication with parties to request additional information, answer questions, or offer therapeutic suggestions. In embodiments, the secure backend data architecture summarizes user information (plots, charts, event logs) concerning progress and outcomes to the care team. Additionally, it flags potential issues based on the results. It also allows healthcare providers to set reminders for remote users and schedule questionnaires. Access is secure, restricted, and logged. All data and logs are encrypted in flight and on the back-end database. In this manner, security and regulatory compliance are attained.

The system provides a strong foundation that can be easily scaled to address remote healthcare monitoring during a mental health crisis, as well as future mental health needs. To perform mental health monitoring a variety of types and combinations of biological signals may need to be captured. The reference design hardware technology (e.g., as may be provided by Maxim Integrates) can collect a wide array of biological signals including HR, HRV, motion, respiratory rate, temperature, and SpO2. The system also may leverage a secondary reference design (e.g., as may be obtained from Philips Research) that integrates electrodermal activity. Coupled with the biological signal front end hardware, the secure mechanism for cloud data transmission and storage facilitates remote data collection, with algorithms to scale and reduce data living both on a smart phone application, as well as in the cloud.

In embodiments, the combination of one or more biological signals to be evaluated may evolve as data becomes available. This aligns with a major technical innovation of the system, to adaptively change the combinations and types of biological signals that can be recorded. A baseline for these indicators can be established before stress events appear. For example, a good model of a user's body temperature and level of tiredness at various times during the day may be obtained. The system can then verify the authenticity of detected events (e.g., coughing, tiredness), and ask for indicators of other symptoms (like gastrointestinal issues) on a regular basis. If is the system detects any indication of a mental health crisis, the individual can be put in touch with frontline telehealth providers or provided with just-in-time and other digital therapies such as videos, games, or interactive content.

In embodiments, the system involves one or more biosensor devices which generate the data by monitoring the biological property of the individual. Methods and operations of the system may be performed, in whole or in part, by a mobile device operably connected with the one or more biosensor devices. In embodiments, the mobile device may include appropriate hardware necessary or sufficient for performing the method in whole or in part, as would be understood by a person having ordinary skill in the art. Exemplary hardware may include a processor, a non-transitory machine-readable medium, a speaker, a microphone, and a human-readable display. In embodiments, one or more logics may be stored on the non-transitory machine-readable medium of the mobile device and executed by the processer of the mobile device when the method is performed.

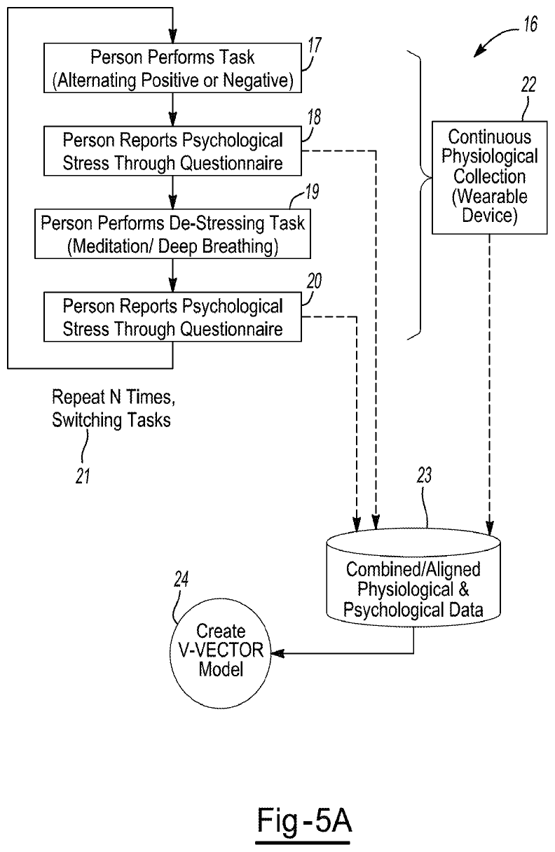

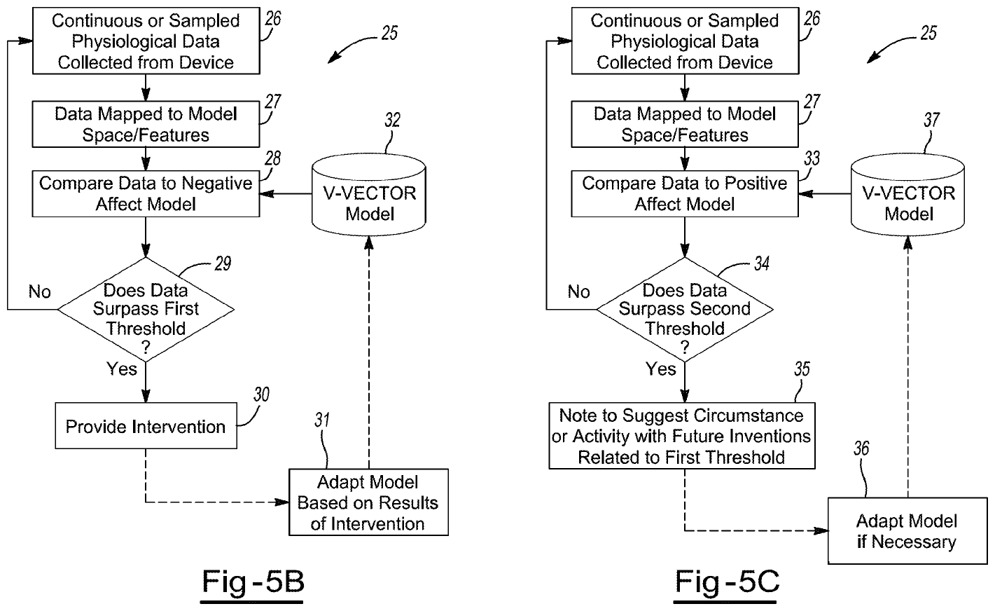

Referring now to FIG. 5A, there is depicted a diagram of an exemplary method of training a device or system in a controlled environment, which may configure the device or system for monitoring an individual for stress in an uncontrolled environment. A method 16 of training the device or system in the controlled environment involves a plurality of steps, all or a portion of which may be performed in the order shown, or in a different order, without departing from the scope of the system. The method 16 may involve a person performing a task 17, which may alternate between positive experience tasks and negative experience tasks, while being continuously monitored 22 to obtain physiological data such as data from one or more biosensors. The person reports their task-based psychological stress 18 via a suitable communication such as a questionnaire, and the person's task-based psychological stress is correlated with the task(s) in the form of a data structure comprising combined and/or aligned physiological and psychological data 23. The person may then perform a de-stressing task 19, such as meditation or breathing exercises, and then after de-stressing, may report their non-task-based psychological stress 20. The non-task-based psychological stress is correlated with the observed physiological data and added to the data structure 23. The task-based training steps (17, 18, 19, 20) may be repeated any number of times, and may involve changing or switching tasks 21 in subsequent iterations of the task-based training steps. The data structure may be used to create a V-vector model or diagram 24 for each observed individual, and the baseline or valence axes are computed based on physiological and psychological data of the data structure 23.

In embodiments, the controlled conditions may include a laboratory setting, a medical setting, or another setting which enables controlled observation of the individual. However, in embodiments, the controlled conditions may include any setting, and the task-based training may be performed as a functionality of a device or a system of the system. As a non-limiting example, delivery of a positive task to the individual may involve displaying a positive image on a computer device, such as the individual's computer, tablet, or smartphone. Similarly, as another non-limiting example, delivery of a negative task to the individual may involve playing a disliked song through an audio functionality of the computer device. In this manner, method 16 may be performed anywhere and at any time.

In embodiments, the trained device or system may benefit the individual through improved stress detection and classification for the individual. In addition, or in the alternative, such trained devices or systems may benefit other users as well, for example, by constructing or updating a model for all or a subset of users of the system. The subset of users may include, for example, other users who exhibit similar responses to a set of tasks or stimuli. Because an individual's V-vector model may be at least partially determined by background or demographic information, such as country of residence, the subset of users may include geographic subsets, socioeconomic subsets, or other subsets with suitable characteristics.

Referring now to FIG. 5A, there is depicted a diagram of a method or operation for monitoring the individual for stress in an uncontrolled environment, and for evaluating detected stress against at least a first threshold for negative stress. A method 25 of monitoring the individual for stress in the uncontrolled environment may be performed as part of operations of a device or system of the system, may be performed as part of a procedure performed by one or more persons such as researchers or caretakers, or a combination thereof. The method 25 involves a plurality of steps, all or a portion of which may be performed in the order shown, or in a different order, without departing from the scope of the system.

In embodiments, the method 25 comprises collecting 26 physiological data points from a device or system of the system, whether continuously or discretely (e.g., sampled). The data is mapped 27 to the space/features of the V-Vector model and compared 28 to the V-vector model 32 wherein the V-vector model 32 may involve at least a first threshold. If all or a portion of the data is above the first threshold 29 (e.g., a negative stress threshold), then the device or system may intervene 30 with the individual. If the data does not surpass the threshold 29, the device or system may continue collecting 26 physiological data. After intervening, the results of the intervention may be used to adapt and/or update 31 the model or V-vector model 32, which may be used in subsequent comparisons 28 between observed data and the V-vector model 32 and the threshold(s).

Referring now to FIG. 5B, there is depicted a diagram of a method or operation of monitoring the individual for stress in the uncontrolled environment, and for evaluating detected stress against at least a second threshold for positive stress. The method 25 may, in addition or in the alternative, may map data 27 to the space/features of the V-Vector model and compare 33 the data to the V-vector model 37 wherein the V-vector model 37 may involve at least a second threshold. If all or a portion of the data is above the second threshold 34 (e.g., a positive stress threshold), then the device or system may record information 35 related to the stress level and/or make a note to suggest an activity or circumstance associated with the positive stress with one or more future interventions related to the first (negative stress) threshold. If the data does not surpass the threshold 34, the device or system may continue collecting 26 physiological data. After recording 35, the information associated with the positive stress may be used to adapt and/or update 36 the model or V-vector model 37, which may be used in subsequent comparisons 33 between observed data and the V-vector model 37 and the threshold(s).

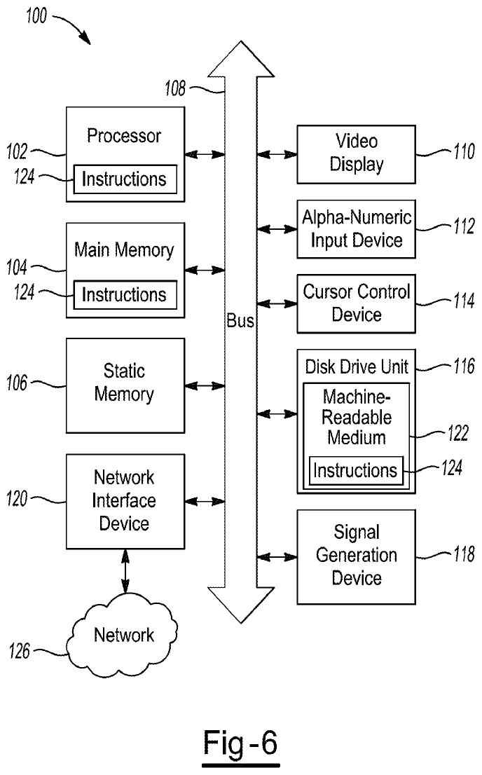

Referring now to FIG. 6, there is depicted a block diagram of a machine in the example form of a computer device or system 100 with which instructions 124 may be executed to cause the machine to perform any one or more of the operations and/or methodologies of the system, in whole or in part, individually or in combination, in any order of events or steps. In embodiments, the machine operates as a standalone device, or can be connected (e.g., networked) to other machines. In a networked deployment, the machine can operate in the capacity of a server or a client machine in server-client network environment, or as a peer machine in a peer-to-peer (or distributed) network environment. The machine can be a personal computer (PC), a tablet PC, a set-top box (STB), a personal digital assistant (PDA), a cellular telephone, a web appliance, a network router, switch, or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein.

The example computer system 100 includes a processor 102 (e.g., a central processing unit (CPU), a graphics processing unit (GPU), or both), a main memory 104, and a static memory 106, which communicate with each other via a bus 108. The computer system 100 can further include a video display 110 (e.g., a liquid crystal display (LCD) or a cathode ray tube (CRT)). The computer system 100 also includes an alpha-numeric input device 112 (e.g., a keyboard or a touch-sensitive display screen), a user interface (UI) navigation (or cursor control) device 114 (e.g., a mouse), a disk drive unit 116, a signal generation device 118 (e.g., a speaker), and a network interface device 120.

The disk drive unit 116 includes a machine-readable medium 122 on which are stored one or more sets of data structures and instructions 124 (e.g., software) embodying or utilized by any one or more of the methodologies or functions described herein. The instructions 124 can also reside, completely or at least partially, within the main memory 104 or within the processor 102, or both, during execution thereof by the computer system 100, with the main memory 104 and the processor 102 also constituting machine-readable media.

While the machine-readable medium 122 is shown in an example embodiment to be a single medium, the term “machine-readable medium” can include a single medium or multiple media (e.g., a centralized or distributed database, and/or associated caches and servers) that store the one or more instructions 124 or data structures. The term “machine-readable medium” shall also be taken to include any tangible medium that is capable of storing, encoding, or carrying instructions 124 for execution by the machine and that cause the machine to perform any one or more of the methodologies of the present disclosure, or that is capable of storing, encoding, or carrying data structures utilized by or associated with such instructions 124. The term “machine-readable medium” shall also be taken to include, but not be limited to, solid-state memories, and optical and magnetic media. Specific examples of machine-readable media 122 include non-volatile memory, including by way of example semiconductor memory devices, e.g., erasable programmable read-only memory (EPROM), electrically erasable programmable read-only memory (EEPROM), and flash memory devices; magnetic disks such as internal hard disks and removable disks; magneto-optical disks; and CD-ROM and DVD-ROM disks.

The instructions 124 can be transmitted or received over a communication network 126 using a transmission medium. The instructions 124 can be transmitted using the network interface device 120 and any one of a number of transfer protocols well known to the skilled artisan (e.g., HTTP). Examples of communication networks include a local area network (LAN), a wide area network (WAN), the Internet or world wide web, mobile telephone networks, plain old telephone (POTS) networks, and wireless data networks (e.g., Wi-Fi and WiMax networks). The term “transmission medium” shall be taken to include any intangible medium which is capable of storing, encoding, or carrying instructions 124 for execution by the machine, and includes digital or analog communications signals or other intangible media to facilitate communication of such software.

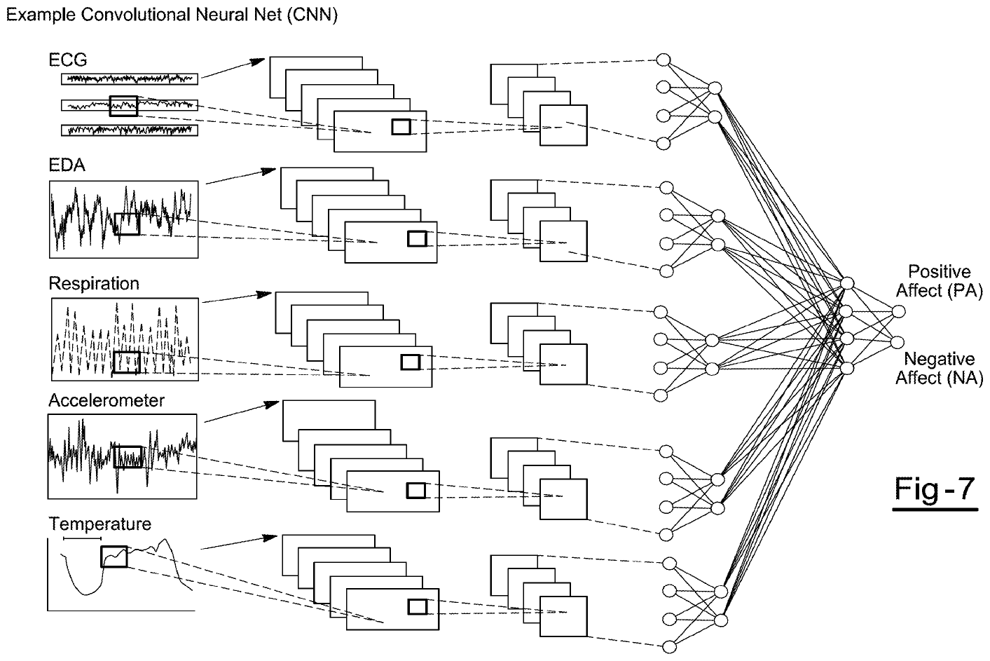

Referring now to FIG. 7, there is depicted a diagram of an exemplary embodiment of the system that utilizes biosignal data (e.g., ECG, EDA, respiration, accelerometer, temperature, or any combination thereof) and a CNN for classifying events as being positive affect (PA) or negative affect (NA). In embodiments, the quantitative processed features from the wearable device may be used to build a physiological stress model. Additionally, simple machine learning algorithmic classifiers, such as support vector machine(s) and/or random forest(s), and more complex models if needed, such as a multi-layer NN network, may be utilized. In embodiments, a self-learning optimizer such as the Tree-Based Pipeline Optimization Tool (TPOT) may be used to apply multiple preprocessing steps and machine learning algorithms.

It may be more effective to integrate with an individual's perceived distress to provide greater resolution to changing anxiety levels. In embodiments, one implementation of the system would be to model the accumulation and decay of stress over time, of either positive stress or negative stress, or both. Another implementation would be to model the stress as two continuous regressions. One regression would be the level of stress (from none to high), and another regression would be the sign or polarity. In embodiments, it is possible to accurately model when the subject is in one of three states from laboratory data (including, for example, a calming task, a distress-causing task, and a eustress-causing task).

The foregoing descriptions of specific embodiments of the system have been presented for purposes of illustration and description. They are not intended to be exhaustive or to limit the system to the precise forms disclosed, and modifications and variations are possible in view of the above teaching. The exemplary embodiments were chosen and described to best explain the principles of the system and its practical application, to thereby enable others skilled in the art to best utilize the system and its embodiments with modifications as suited to the use contemplated.

systemsystem With respect to the description provided herein, it is submitted that the optimal features of the system include variations in size, materials, shape, form, function and manner of operation, assembly, and use. All structures, functions, and relationships equivalent or essentially equivalent to those disclosed are intended to be encompassed by the system. It is noted that the terms “substantially” and “about” may be utilized herein to represent the inherent degree of uncertainty that may be attributed to any quantitative comparison, value, measurement, or other representation. Any values that may be modified by such terminology are also part of the teachings herein. For example, if a teaching recited “about 10,” the skilled person should recognize that the value of 10 is also contemplated.

These terms are also utilized herein to represent the degree by which a quantitative representation may vary from a stated reference without resulting in a change in the basic function of the subject matter at issue.

As used herein, unless otherwise stated, the teachings envision that any member of a genus (list) may be excluded from the genus; and/or any member of a Markush grouping may be excluded from the grouping.

Unless otherwise stated, any numerical values recited herein include all values from the lower value to the upper value in increments of one unit provided that there is a separation of at least 2 units between any lower value and any higher value. As an example, if it is stated that the amount of a component, a property, or a value of a process variable such as, for example, temperature, pressure, time and the like is, for example, from 1 to 90, preferably from 20 to 80, more preferably from 30 to 70, it is intended that intermediate range values such as (for example, 15 to 85, 22 to 68, 43 to 51, 30 to 32 etc.) are within the teachings of this specification. Likewise, individual intermediate values are also within the present teachings. For values which are less than one, one unit is considered to be 0.0001, 0.001, 0.01 or 0.1 as appropriate. These are only examples of what is specifically intended and all possible combinations of numerical values between the lowest value and the highest value enumerated are to be considered to be expressly stated in this application in a similar manner. As can be seen, the teaching of amounts expressed as “parts by weight” herein also contemplates the same ranges expressed in terms of percent by weight. Thus, an expression in the Detailed Description of a range in terms of at “‘x’ parts by weight of the resulting polymeric blend composition” also contemplates a teaching of ranges of same recited amount of “x” in percent by weight of the resulting polymeric blend composition.”

Unless otherwise stated, all ranges include both endpoints and all numbers between the endpoints. The use of “about” or “approximately” in connection with a range applies to both ends of the range. Thus, “about 20 to 30” is intended to cover “about 20 to about 30”, inclusive of at least the specified endpoints.

The term “consisting essentially of” to describe a combination shall include the elements, ingredients, components or steps identified, and such other elements ingredients, components or steps that do not materially affect the basic and novel characteristics of the combination. The use of the terms “comprising” or “including” to describe combinations of elements, ingredients, components or steps herein also contemplates embodiments that consist essentially of, or even consist of the elements, ingredients, components or steps.

Plural elements, ingredients, components or steps can be provided by a single integrated element, ingredient, component or step. Alternatively, a single integrated element, ingredient, component or step might be divided into separate plural elements, ingredients, components or steps. The disclosure of “a” or “one” to describe an element, ingredient, component or step is not intended to foreclose additional elements, ingredients, components or steps. All references herein to elements or metals belonging to a certain Group refer to the Periodic Table of the Elements published and copyrighted by CRC Press, Inc., 1989. Any reference to the Group or Groups shall be to the Group or Groups as reflected in this Periodic Table of the Elements using the IUPAC system for numbering groups.

While particular embodiments have been illustrated and described herein, it should be understood that various other changes and modifications may be made without departing from the spirit and scope of the claimed subject matter.

Moreover, although various aspects of the claimed subject matter have been described herein, such aspects need not be utilized in combination.

It is therefore intended that the appended claims (and/or any future claims filed in any corresponding application) cover all such changes and modifications that are within the scope of the claimed subject matter.

1. A method for a system comprising a non-transitory machine-readable medium storing instructions that, when executed by at least one processor, cause the at least one processor to perform operations for mental health assessment, the operations comprising the steps of:
determining at least a first set of data which corresponds to at least one physiological parameter of an individual, an environment of the individual, an interaction between the individual and the environment, or any combination thereof;
evaluating said least a first set of data; and
detecting the presence and magnitude of positive and negative emotional valences of the individual based on the evaluation of at least the first set of data.

2. The method for a system of claim 1, wherein the operations further comprise the steps of:
determining a second set of data which corresponds to which corresponds to at least one previous exposure of the individual to at least one known positive stressor, at least one known negative stressor, or any combination thereof;
evaluating said second set of data; and
wherein the evaluation of the first and second sets of data yields the positive and negative emotional valences of the individual.

3. The method for a system of claim 1, wherein the operations further comprise:
querying the individual with a conversational agent of the instructions to produce at least one query result;
evaluating the at least one query result to assist the operations with the detecting the positive and negative emotional valences; and
wherein the operations further comprise:
determining a magnitude of each of the positive and negative emotional valences; and
placing at least one data point on a V-vector model based on the magnitudes.

4. The method for a system of claim 1, wherein the operations further comprise:
determining a magnitude of each of the positive and negative emotional valences; and
placing at least one data point on a V-vector model based on the magnitudes.

5. The method for a system of claim 4, wherein the operations further comprise:
providing an intervention to the individual if the negative emotional valence surpasses a first threshold of the V-vector model and recording information related to a stress level of the individual if the positive emotional valence surpasses a second threshold of the V-vector model.

6. A system for mental health assessment, the system for mental health assessment comprising:
at least one processor; and
at least one non-transitory machine-readable medium storing instructions that, when executed by the at least one processor, cause the at least one processor to perform operations for mental health assessment, the operations comprising the steps of:
determining at least a first set of data which corresponds to at least one physiological parameter of an individual, an environment of the individual, an interaction between the individual and the environment, or any combination thereof;
evaluating said least a first set of data; and
and
detecting positive and negative emotional valences of the individual based on the evaluation of that data.

7. The system of claim 6, wherein the operations further comprise:
evaluating a second set of data which corresponds to at least one previous exposure of the individual to at least one known positive stressor, at least one known negative stressor, or any combination thereof;
wherein the evaluation of the first and second sets of data yields the positive and negative emotional valences of the individual.

8. The system of claim 6, wherein the operations further comprise:
querying the individual with a conversational agent of the instructions to produce at least one query result; and
evaluating the at least one query result to assist the operations with the detecting the positive and negative emotional valences.

9. The system of claim 6, wherein the operations further comprise:
determining a magnitude of each of the positive and negative emotional valences; and
placing at least one data point on a V-vector model based on the magnitudes.

10. The system of claim 9, wherein the operations further comprise:
providing an intervention to the individual if the negative emotional valence surpasses a first threshold of the V-vector model.

11. The system of claim 9, wherein the operations further comprise:
recording information related to a stress level of the individual if the positive emotional valence surpasses a second threshold of the V-vector model.

12. A method for assessing mental health of an individual, comprising:
determining at least a first set of data which corresponds to at least one physiological parameter of the individual, an environment of the individual, an interaction between the individual and the environment, or any combination thereof
evaluating said at least a first set of some data;
detecting positive and negative emotional valences of the individual based on the evaluation of at least the first set of data; and
evaluating a second set of data which corresponds to at least one previous exposure of the individual to at least one known positive stressor, at least one known negative stressor, or any combination thereof;
wherein the evaluation of the first and second sets of data yields the positive and negative emotional valences of the individual.

13. The method of claim 12, further comprising querying the individual to assist with the detecting the positive and negative emotional valences.

14. The method of claim 12, further comprising:
determining a magnitude of each of the positive and negative emotional valences; and
placing at least one data point on a V-vector model based on the magnitudes.

15. The method of claim 14, further comprising:
providing an intervention to the individual if the negative emotional valence surpasses a first threshold of the V-vector model and/or recording information related to a stress level of the individual if the positive emotional valence surpasses a second threshold of the V-vector model.
